(19) United States
(12) Patent Application Publication
(10) Pub. No.: US 2008/0167526 A1
(43) Pub. Date: Jul. 10, 2008

(54) Non-Occlusive, Laterally-Constrained Injection Device

(76) Inventors: Justin M. Crank, Maple Grove, MN (US); Sidney F. Hauschild, St. Paul, MN (US)

Correspondence Address: AMS RESEARCH CORPORATION, 10700 BREN ROAD WEST, MINNETONKA, MN, 55343, US

(21) Appl. No.: 11/970,343

(22) Filed: Jan. 7, 2008

Related U.S. Application Data
(60) Provisional application No. 60/883,879, filed on Jan. 8, 2007.

(51) Int. Cl. A61B 1/012; A61B 1/307

(52) U.S. Cl. 600/135; 600/156

(57) ABSTRACT

A flexible cytoscope having an injection device with a cross-sectional profile allowing for irrigation flow without the risk of buckling or lateral deflection of the injection device. The cross-sectional profile is configured such that the injection device is constrained by the cytoscope lumen. The cross-sectional profile of the injection device can be formed integrally with the injection device or can alternatively comprise a secondary structure that is attached about an exterior portion of the injection device. The cross-sectional profile can be present at intervals along the length of the injection device or can extend the entire length of the injection device. The injection device can be configured such that the cross-sectional profile centers the injection device within the cytoscope lumen or in an off-set or non-centered position depending upon the treatment being performed.

### PRIORITY CLAIM

The present application claims priority to U.S. Provisional Application Ser. No. 60/883,879, filed Jan. 8, 2007 and entitled, “NON-OCCLUSIVE, LATERALLY-CONSTRAINED INJECTION TUBE”, which is hereby incorporated by reference in its entirety.

### FIELD OF THE INVENTION

The present invention relates generally to minimally invasive medical access devices delivering a therapeutic treatment to a treatment site within a patient's body. More specifically, the present invention relates to an injection device residing within a working channel of a cytoscope wherein the injection device has a cross-sectional profile that interfaces with the cytoscope so as to resist buckling or lateral deflection while providing an irrigation channel for the return of irrigation fluid.

### BACKGROUND OF THE INVENTION

A wide variety of medical treatments are at least partially performed through the delivery and introduction of therapeutic compositions to a treatment location. In home or outpatient settings, typical delivery methods can comprise oral delivery, via liquid or solid forms, as well as a variety of inhalant style devices. In clinical or hospital settings, therapeutic fluids can be injected using needle based or in some minimally invasive procedures, the therapeutic fluid can be delivered through a tubular device such as a catheter or endoscope based system.

One common tube-based device for urological use is commonly referred to as a cytoscope. Cytoscopes are designed to examine the bladder, lower urinary tract and prostate gland. In addition to examination, cytoscopes can be used to deliver treatment and/or treatment devices to identified treatment areas. Cytoscopes are available in both rigid and flexible configurations and typically differ only in respect to their method of insertion. Flexible cytoscopes are especially advantageous in that the patient is not required to assume the lithotomy position for the length of a procedure, which can typically require anywhere from 10-40 minutes to complete.

Prior to use, the cytoscope is lubricated and then passed through the urethra and into the bladder. Once the cytoscope is positioned within the bladder, fluid is injected through the cytoscope and into the bladder so as to inflate the bladder whereby the urologist can examine the entire bladder wall. Depending upon the procedure type, instruments such as, for example, a tiny basket, grasper, brush or forceps can be inserted through the cytoscope to remove stones, gather tissue samples or inject x-ray dyes. Following the procedure, the cytoscope is removed.

While flexible cytoscopes can be advantageous to a patient's comfort during these procedures, the flexible nature of the cytoscope can be a disadvantage due to the potential for buckling or lateral deflection of the cytoscope. As such, it would be advantageous to have a flexible cytoscope that combines the comfort advantages of the flexible cytoscope with the strength and positioning assurance of a rigid cytoscope.

### SUMMARY OF THE INVENTION

The present invention comprises a flexible cytoscope having an injection device or device located within a working channel of the cytoscope. The injection device can comprise a cross-sectional profile that interfaces with the cytoscope so as resist buckling or lateral deflection of the injection device while maintaining an open portion within the working channel allowing for irrigation flow. The cross-sectional profile is configured such that the injection device is in contact with the walls at multiple points such that the position and orientation of the injection device is constrained by the cytoscope. The cross-sectional profile of the injection device can be formed integrally with the injection device or can alternatively comprise a secondary, exterior structure that is attached about an exterior portion of the injection device. The injection device can be configured such that the cross-sectional profile centers the injection device within the cytoscope lumen or in an off-set or non-centered position depending upon the treatment being performed. The injection device can be fabricated so as to have an injection lumen located at a distal, treatment end of the injection device or alternatively, the injection device can comprise a side orifice within a side wall of the injection device.

In one aspect, the present application is directed to a flexible cytoscope comprising an injection device with a selected cross-sectional profile such that a cytoscope lumen constrains the injection device so as to reduce the potential for buckling and/or lateral deflection of the injection device. The selected cross-sectional profile can orient the injection device in either a centered or non-centered off-set position within the cytoscope lumen. The selected cross-sectional profile can further define an irrigant return channel within a working channel of the cytoscope. In some embodiments, the cross-sectional profile can provide for a single injection lumen or, alternatively, two or more injection lumens located at a distal end location or alternatively, along a side wall of the injection device. The injection device can be fabricated such that the cross-sectional profile is integrally formed as part of the injection device or alternatively, the selected tube cross-section can be formed through the application of an exterior constraining member around a perimeter portion of the injection device.

In another aspect, the present application is directed to a method for preventing buckling and/or lateral deflection of an injection device introduced through a cytoscope during a urological procedure. Generally, the method can comprise providing an injection device having a selected injection cross-sectional profile intended to contact the walls of the cytoscope at a plurality of locations such that the injection device is positionally constrained within a working channel of the cytoscope. In some embodiments, the method can comprise fabricating the injection device such that an exterior projecting member is attached around an exterior portion of the injection device so as to define the selected injection cross-sectional profile. The exterior projecting member can be integrally formed with the injection device or can be independently added to a conventional injection tube so as to define the selected injection cross-sectional profile. In some embodiments, the method can further comprise returning an irrigant flow through a return channel defined by the interaction of the selected cross-sectional profile within the working channel of the cytoscope.

In yet another aspect, the present application is directed to a urological treatment system comprising a flexible cytoscope having an injection device with a selected cross-sectional profile such that the injection device is constrained within a working channel of the cytoscope thereby reducing the potential for buckling and/or lateral deflection of the injection device. The injection device can comprise one or more injection lumens and may comprise additional treatment components including, for example, fiber optic lights and/or an objective lens. The urological treatment system can further comprise an imaging apparatus for confirming the position of the flexible cytoscope within the patient, and to confirm the positioning of an injection lumen prior to administering a therapeutic fluid or similar treatment.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is an illustration of a flexible cytoscope positioned within a patient's urogenital tract.

FIG. 2 is a perspective view of a distal portion of a flexible cytoscope including an injection device according to an embodiment of the present disclosure.

FIG. 2a is a perspective view of a distal portion of a flexible cytoscope including an injection device with a side delivery orifice according to an embodiment of the present disclosure.

FIG. 3 is an end view of a distal portion of a flexible cytoscope including an injection device according to an embodiment of the present disclosure.

FIG. 4 is an end view of a distal portion of a flexible cytoscope including an injection device according to an embodiment of the present disclosure.

FIG. 5 is an end view of a distal portion of a flexible cytoscope including an injection device according to an embodiment of the present disclosure.

FIG. 6 is a partially hidden side view of a portion of a flexible cytoscope including an injection device according to an embodiment of the present disclosure.

FIG. 6a is a section view of the flexible cytoscope of FIG. 6 taken at line 6a-6a of FIG. 6.

FIG. 7 is a partial hidden side view of a portion of a flexible cytoscope including an injection device according to an embodiment of the present disclosure.

FIG. 7a is a section view of the flexible cytoscope of FIG. 7 taken at line 7a-7a of FIG. 7.

FIG. 8 is a partially hidden side view of a portion of a flexible cytoscope including an injection device according to an embodiment of the present disclosure.

FIG. 8a is a section view of the flexible cytoscope of FIG. 8 taken at line 8a-8a of FIG. 8.

FIG. 9 is a partially hidden side view of a portion of a flexible cytoscope including an injection device according to an embodiment of the present disclosure.

FIG. 9a is a section view of the flexible cytoscope of FIG. 9 taken at line 9a-9a of FIG. 9.

FIG. 10 is an end view of a flexible cytoscope including an injection device according to an embodiment of the present disclosure.

FIG. 11 is an end view of a flexible cytoscope including an injection device according to an embodiment of the present disclosure.

FIG. 12 is an end view of a flexible cytoscope including an injection device according to an embodiment of the present disclosure.

FIG. 13 is an end view of a flexible cytoscope including an injection device according to an embodiment of the present disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

### DETAILED DESCRIPTION OF THE DRAWINGS

In the following detailed description of the present invention, numerous specific details are set forth in order to provide a thorough understanding of the present invention. However, it will be obvious to one skilled in the art that the present invention may be practiced without these specific details. In other instances, well-known methods, procedures, and components have not been described in detail so as to not unnecessarily obscure aspects of the present invention.

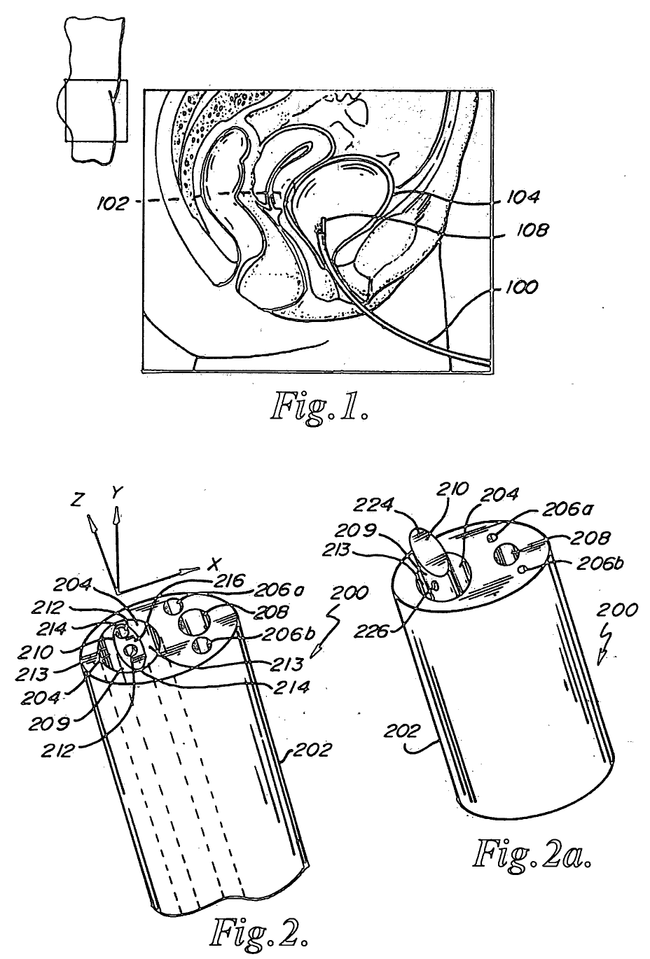

Referring to FIG. 1, a flexible cytoscope 100 of the prior art can be used to deliver treatment to a desired location in a patient's body 102 such as, for example, a patient's urinary bladder 104. Generally, flexible cytoscope 100 can comprise a length of polymeric tubing 106 having a distal treatment end 108. In some embodiments, flexible cytoscope 100 can delivery a therapeutic fluid to the treatment location through the polymeric tubing 106 or alternatively, the polymeric tubing can be use to provide access for medical instruments and/or tools such as, for example, a fiber optic scope and/or light to assist in diagnosing and/or treating tissue.

In positioning the flexible cytoscope at a treatment location, it will be understood that a medical professional frequently employs a medical imaging system such as, for example, computer axial tomography (CAT), magnetic resonance imaging (MRI), or in the case of treatment of a prostate gland, the preferred imaging means is transrectal ultrasound (TRUS) so as to achieve the desired position of the distal treatment end 108. Through the use of a medical imaging system, a medical professional can verify that the distal treatment end 108 is properly positioned for delivering therapy at the treatment location.

Referring to FIG. 2, a flexible cytoscope 200 of the present invention can comprise a length of polymeric tubing 202 having a working channel 204 and one or more treatment tools such as, for example, fiber optic lights 206a, 206b and an objective lens 208. Located within working channel 204 is an injection device 210, such as a needleless injector (e.g., jet injector), that can include one or more injection lumens 212. Injection device 210 is configured so as to have a device body 109 with a cross-sectional profile that does not fully occupy the working channel 204 so as to define an open channel 213. Injection device 210 simultaneously contacts the polymeric tubing 202 at a plurality of contact locations 214, for example see FIG. 4, so as to maintain a desired orientation within the working channel 204 and to provide lateral support to the flexible cytoscope 200.

Polymeric tubing 202 and injection device 210 are preferably fabricated of medical grade polymers and copolymers. In some embodiments, polymeric tubing 202 and injection device 210 can be molded of the same polymer so as to promote maximum compatibility and similar performance characteristics. Depending upon the treatment application, polymeric tubing 202 and/or injection device 210 can be fabricated with high strength polymers including, for example, polyimide, polyetherimide available from General Electric under the trade name Ultem® and linear aromatic polymers such as PEEK™ available from Victrex plc. In some embodiments, the polymeric tubing 202 and/or the injection device 210 can be reinforced through the inclusion of materials including nano-particles, clays and/or glass within the polymer. Alternatively, polymeric tubing 202 and injection device 210 can be reinforced with one or more polymers such as, for example, tubes braided with Kevlar or other high-strength polymers. In some embodiments, the polymeric tubing 202 and/or injection device 210 can be fabricated so as to have a burst strength exceeding at least about 2,000 psi and in some embodiments, having a burst strength within a range of about 2,000 psi to about 5,000 psi.

In use, flexible cytoscope 200 can be positioned for treatment as previously described with the cytoscopes of the prior art. As the injection device 210 is slidably introduced into the working channel 204, the cross-section of the injection device 210 and more specifically, the contact locations 214 constrain the orientation and positioning of the injection device 210 such that the injection device 210 cannot buckle within the working channel 204. As the injection device 210 cannot buckle within the working channel 204, open channel 213 remains unobstructed so as to accommodate irrigant flow to a treatment location. As injection device 210 is advanced through the working channel 204, the injection device 210 can be oriented such that the preferred axis of bending for the injection device 210 matches the preferred axis of bending of the cytoscope 200 so as to resist twisting of the injection device 210 and to maintain the desired orientation of the injection device 210. For instance, injection device 210 can be oriented such that the preferred axis of bending for both the injection device 210 and the cytoscope 200 is the x-z plane as illustrated in FIG. 2.

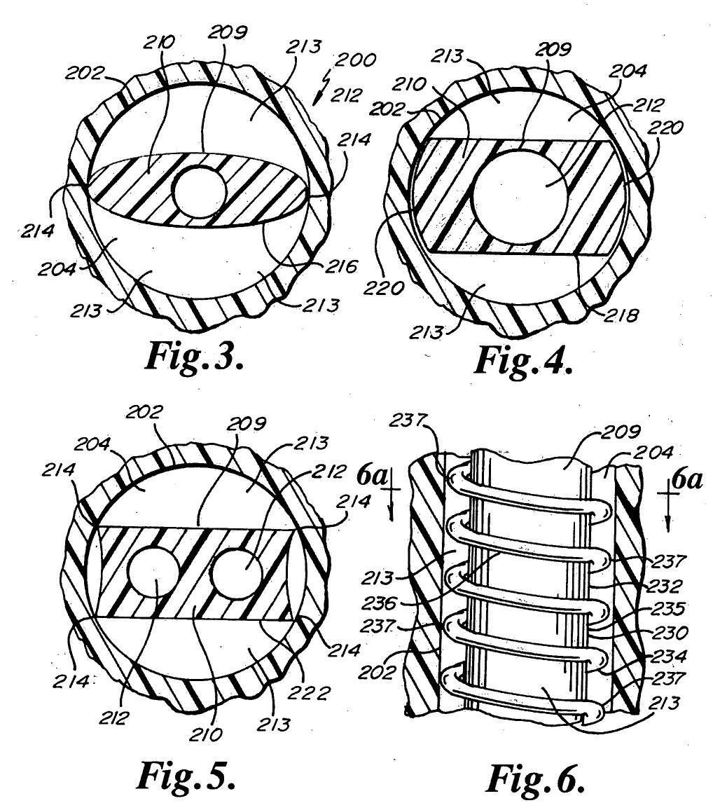

Device body 209 can comprise a variety of cross-sectional arrangements that similarly constrain the orientation and positioning of the injection device 210 within the working channel 204 as well as preventing bending and twisting of the injection device 210. For instance, injection device 210 can comprise an oval cross-sectional profile 216 as illustrated in FIG. 2 with a pair of injection lumens 212 or with a single injection lumen 212 as illustrated in FIG. 3. Oval cross-sectional profile 216 is in physical contact with the polymeric tubing 202 at two contact locations 214. As illustrated in FIG. 4, injection device 210 can comprise an arcuate rectangular cross-sectional profile 218 having a single injection lumen 212. Arcuate rectangular cross-sectional profile 218 is in physical contact with polymeric tubing 202 at a pair of arcuate contact locations 220. Referring to FIG. 5, injection device 210 can comprise an inwardly arcuate rectangular cross-sectional profile 222 having a pair of injection lumens 212. Inwardly arcuate rectangular cross-sectional profile 222 is in physical contact with polymeric tubing 202 at four individual contact locations 214.

Referring to FIG. 2a, injection device 210 can be configured such that device body 209 has a closed distal portion 224 with injection lumen 212 oriented as a side delivery orifice 226. Closed distal portion 224 can be extended past a distal end of the cytoscope 200 such that a therapeutic fluid can be aimed and delivered to a treatment location with side delivery orifice 226. Due to the oval cross-sectional profile 216, the injection device 210 is constrained and the orientation and positioning of the injection device 210 is controlled within the working channel 204 while preventing bending and twisting of the injection device 210.

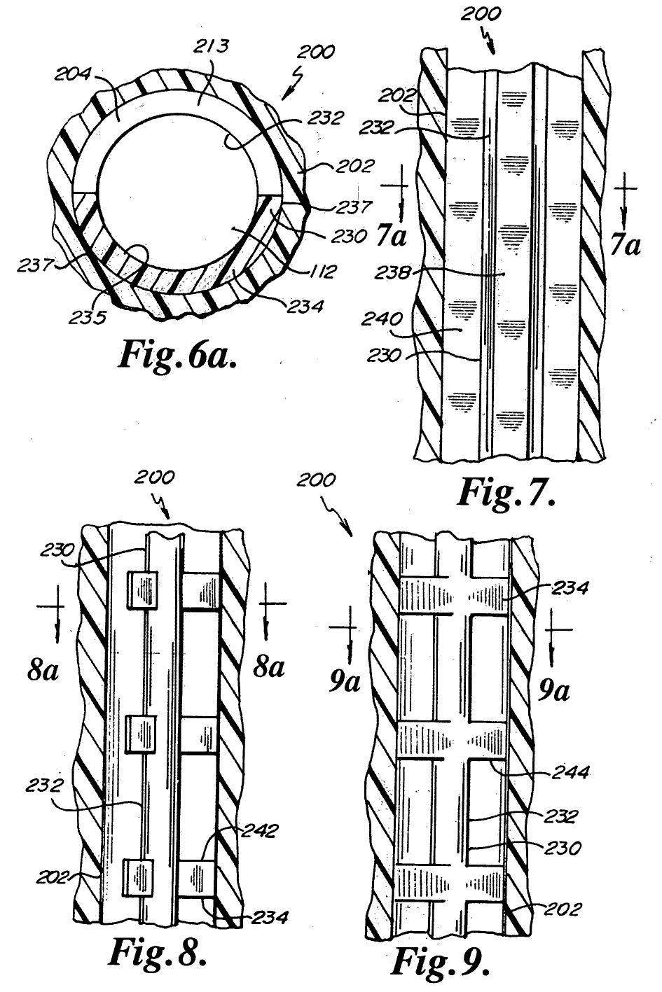

Referring to FIGS. 6 and 6a, another representative embodiment of an injection device 230 can comprise a central injection device 232 and an exterior projection 234 that is in contact with the polymeric tubing 202 so as to constrain the orientation and positioning of the injection device 230 within the working channel 204 as well as preventing bending and twisting of the injection device 230. Central injection device 232 can comprise an injection tube 235 physically resembling cytoscope 200 with the exception of having a smaller diameter. In one representative embodiment, exterior projection 234 can comprise a wrapped coil 236 wrapped around at least a portion of the central injection device 232 within the polymeric tubing 202. Wrapped coil 236 can be formed with suitable medical grade polymers and copolymers. When combined, central injection device 232 and wrapped coil 234 define an injection diameter 236 that is equal to a diameter of the working channel 204 such that wrapped coil 236 contacts the polymeric tubing 202 at a plurality of distinct contact points 237 that are arranged in spiraling relation around the injection tube 235 so as to define open channel 213 within the working channel 204. Wrapped coil 236 can be present along the entire length of injection tube 235 or can be positioned at one or more discreet locations along the length of injection tube 235. Wrapped coil 236 can be integrally formed with injection tube 235 or can alternatively, comprise a distinct component that is positioned around the injection tube 235.

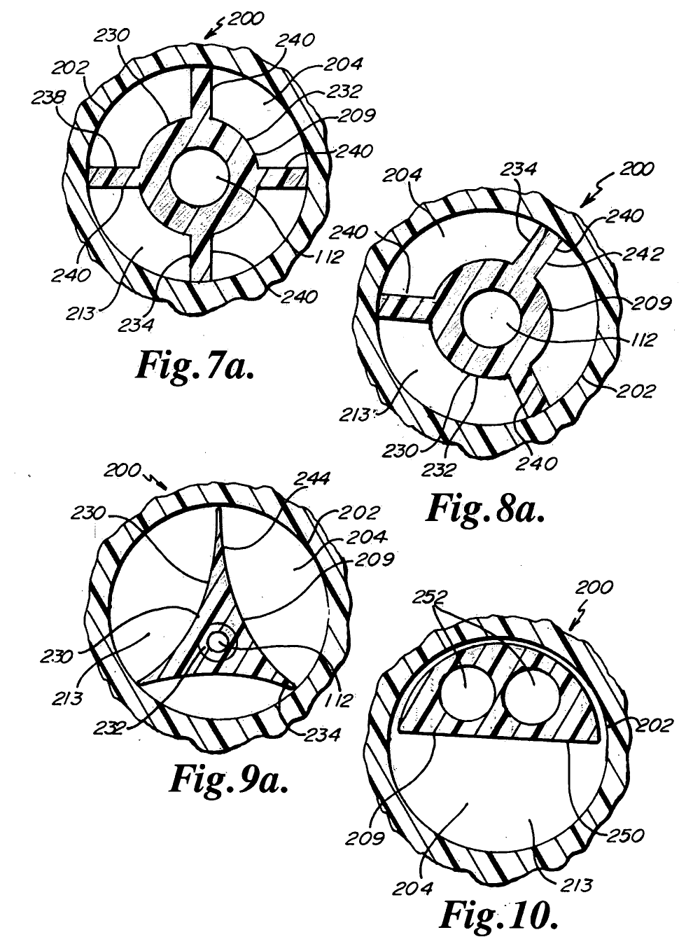

Injection device 230 can utilize a variety of configurations for exterior projection 234 that similarly constrain the orientation and positioning of the injection device 230 within the working channel 204 as well as preventing bending and twisting of the injection device 230. For instance, injection device 230 can comprise a four rib profile 238 as illustrated in FIGS. 7 and 7a. Four rib profile 238 can comprise four individual ribs 240 that extend outward from the central injection device 232 such that each rib contacts the polymeric tubing 202. Each rib 240 can be intermittently spaced along the length of injection device 232 or can extend the entire length of injection device 232. Alternatively, injection device 230 can comprise a three rib profile 242 as illustrated in FIGS. 8 and 8a or a triangular profile 244 as illustrated in FIGS. 9 and 9a. With three rib profile 242 and triangular profile 244, exterior projection 234 contacts the polymeric tubing 202 at three distinct locations. The three rib profile 242 and triangular profile 244 can be intermittently spaced along the length of injection device 232 or can extend the entire length of injection device 232.

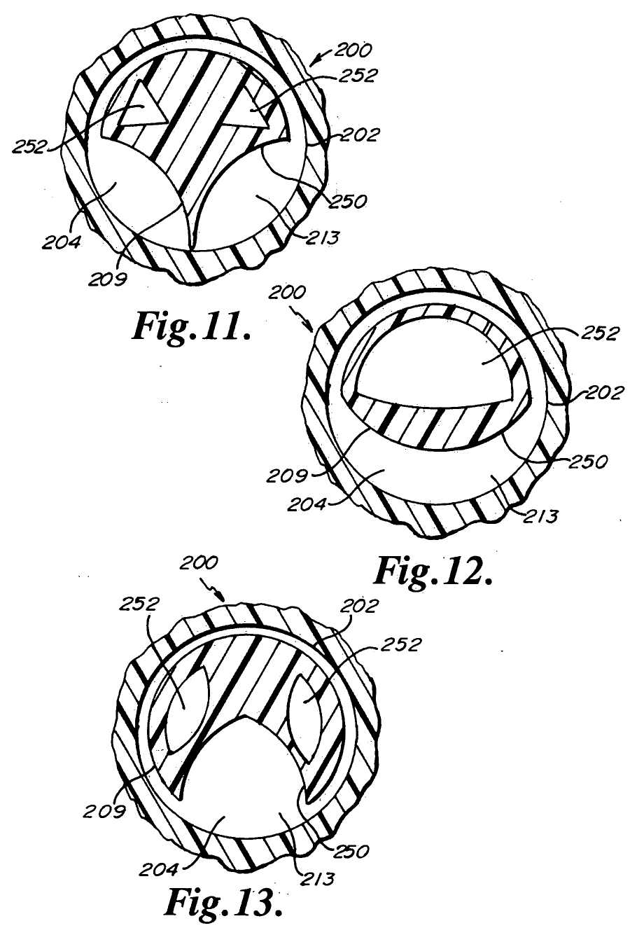

Referring to FIGS. 10, 11, 12 and 13, another representative embodiment of an injection device 250 can comprise one or more injection lumens 252. Injection device 250 can have a cross-sectional profile selected such that the one or more injection lumens are not centered within the injection device 250 and correspondingly not centered within the working channel 204. Arranging the injection lumens 252 in a non-centered location can provide desirable flow characteristics through the working channel 204 and can assist in delivering treatment to a desired location through the injection lumen 252. Injection device 250 includes a cross-sectional profile that is in contact with the polymeric tubing 202 at so as to constrain the orientation and positioning of the injection device 250 within the working channel 204 as well as preventing bending and twisting of the injection device 250.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

1. A flexible cytoscope system comprising:
a flexible cytoscope having a length of tubing including a working channel defined therein; and
an injection device having an injection lumen defined therein, the injection device residing within the working channel to define an open channel, the injection device having a device body defining a device cross-section continuously in contact with the working channel at two or more contact locations so as to provide lateral support to the flexible cytoscope.

2. The flexible cytoscope system of claim 1, wherein the device cross-section is integrally molded with the device body.

3. The flexible cytoscope system of claim 2, wherein the device cross-section is selected from the group consisting of: an oval cross-sectional profile, an arcuate rectangular cross-sectional profile and an inwardly rectangular cross-sectional profile.

4. The flexible cytoscope system of claim 1, wherein the device cross-section comprises an external projection attached to the device body.

5. The flexible cytoscope system of claim 4, wherein the external projection extends continuously along a length of the injection device.

6. The flexible cytoscope system of claim 4, wherein the external projection comprises a wrapped coil around the device body.

7. The flexible cytoscope system of claim 1, wherein the device body includes a distal closed, the device body further including the injection lumen oriented as a side delivery orifice.

8. The flexible cytoscope system of claim 1, wherein the flexible cytoscope and the injection device share a common bending axis.

9. The flexible cytoscope system of claim 1, wherein the injection device includes a plurality of injection lumens.

10. A method of delivering a treatment fluid to a treatment location comprising:
providing an injection device having an injection lumen defined therein, the injection device having a device body defining a device profile cross-section;
positioning the injection device within a working channel of a flexible cytoscope, the device profile cross-section continuously in contact with the working channel at two or more contact locations;
advancing the injection device through the working channel such that injection lumen is positioned proximate a treatment location; and
delivering a therapeutic fluid to treatment location through the injection lumen.

11. The method of claim 10, further comprising:
defining an open channel between the device body and the working channel; and
delivering an irrigation fluid to the treatment location through the open channel.

12. The method of claim 10, further comprising:
positioning the injection lumen proximate the treatment location using a medical imaging system.

13. The method of claim 10, further comprising:
matching a device bend axis of the injection device with a cytoscope bend axis of the cytoscope.

14. A method for preventing obstruction of a flexible cytoscope comprising:
providing an injection device having a device body defining a device cross-sectional profile; and
positioning the injection device within a working lumen of a flexible cytoscope such that the device cross-sectional profile contacts the working lumen in a least two contact locations.

15. The method of claim 14, further comprising:
molding the device cross-sectional profile into the device body.
